(19) United States
(12) Patent Application Publication
(10) Pub. No.: US 2006/0111376 A1
(43) Pub. Date: May 25, 2006

(54) 2,4-Diamino quinazoline and pyridopyrimidine ester derivatives as dihydrofolate reductase inhibitors

(76) Inventors: Anders Hallberg, Uppsala (SE); Malin Graffner-Nordberg, Uppsala (SE); Arne Boman, Uppsala (SE); Elisabeth Seifert, Uppsala (SE)

Correspondence Address: DINSMORE & SHOHL, LLP, 1900 CHEMED CENTER, 255 EAST FIFTH STREET, CINCINNATI, OH, 45202, US

(21) Appl. No.: 10/525,906

(22) PCT Filed: Aug. 27, 2003
(86) PCT No.: PCT/GB03/03714

§ 371 Date: Nov. 14, 2005

(30) Foreign Application Priority Data
Aug. 27, 2002 (GB) 0219897.6

(51) Int. Cl. A61K 31/517; C07D 239/95

(52) U.S. Cl. 514/266.4; 544/291

(57) ABSTRACT

The invention provides novel compounds or the formula II: wherein R₁, R₂, R₃ and R₄ are independently hydrogen of a group that liberates the free amine in vivo, for example —CO-alkyl, preferably —CO—C₁-C₃ alkyl or pivalate; or —COhaloalkyl, preferably —CO—C₁-C₃ haloalkyl, most preferably —CO—C₁-C₃ chloroalkyl; wherein W is: and @ denotes the points of attachment and wherein the ester can be located in either direction; wherein n and m are independently 0-5; wherein one but not both or X and Y can be nitrogen, or X is C-A and/or Y is C—B; wherein A and B are independently selected from hydrogen, alkyl optionally substituted with a halogen, an electron donor group such as amino, alkylamino, dialkylamino or hydroxy, or an electron acceptor group such as nitro, cyano. trihaloalkyl or amido, alkoxy or halogen; and pharmacologically acceptable salts thereof. Provided that when R₁ to R₄ are hydrogen, both X and Y are C—H, n is 1 and —(CH₂)ₙ— is attached to the bridging oxygen of the ester group W, then m cannot be 0 or 1.

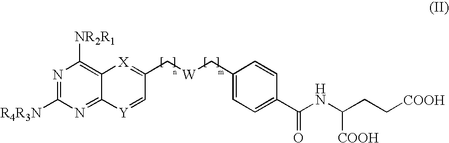

### FIELD OF THE INVENTION

This invention relates to novel esters and salts thereof and the use of these esters as dihydrofolate reductase inhibitors. The compounds in the present invention show improved selectivity relative to cellular reductases and/or improved pharmacokinetic profiles and can be used for the treatment of diseases/conditions which can be therapeutically treated by immuno-modulating or cytostatic compounds, either applied topically, orally or parenterally, or cancer forms be sensitive to methotrexate. The compounds in the present invention can also be used for treating diseases/conditions that involves one or several of the melanocortin receptors. Another area where these compounds can be used involves treatment of nephritis, e.g. IgA nephritis. Other diseases to be treated are inflammatory bowel disease i.e. ulcerative colitis and Crohn's disease, and colorectal cancer, asthma, psoriasis. Pneumocystis carinii pneumonia (PCP), or other serious pulmonary diseases, rheumatoid arthritis (other inflammatory conditions), other fungal infections (vaginal and others), bacterial inflammations, protozoal inflammations, cancer of the urinary bladder, the lung and other cancer types that may be reached from the outside of the body, non-surgical abortions (intrauterin administration), liver transplantation, may be treated, especially in immuno-compromised individuals. Methods of the preparation of such compounds, compositions containing them and novel intermediates therefore are also provided.

### BACKGROUND OF THE INVENTION

Inflammatory bowel disease (IBD) is a general term that includes both utcerative colitis and Crohn's disease, disorders of unknown ethiology that result in inflammation in the gastrointestinal tract. Ulcerative colitis is an inflammatory disease of the large intestine. Ulcers develop in the inner lining, or mucosa, of the colon or rectum, often resulting in diarrhea, blood, and pus. Crohn's disease is an inflammation that extends into the deeper layers of the intestinal wall. It is found most often in the ileum and the first part of the large intestine, known as the ileocecal region.

Ulcerative colitis and Crohn's disease share many symptoms, although they also differ in important ways. Both are chronic diseases characterized by frequent relapses and remissions, and symptoms usually appear in young adults. The most common symptom of both ulcerative colitis and Crohn's disease is diarrhea. Constipation may develop during active flare-ups of both Crohn's disease and ulcerative colitis. Cramps can occur from intestinal contractions caused by inflammation. Fever, fatigue and loss of appetite are often present. Neurologic or psychiatric symptoms may be early signs of Crohn's disease when accompanied by gastrointestinal problems.

Drugs presently on the market cannot cure IBD, but some are effective in reducing the inflammation and accompanying symptoms in up to 80% of patients. Many such drugs are available, including corticosteroids, aspirin-like medications, and drugs that suppress the immune system. The primary goal of drug therapy is to put acute flares into remission and/or prevent relapse. Mesalazine is the common name of the compound 5-aminosalicylic acid or 5-ASA, which inhibits substances in the immune system that cause inflammation.

For very active IBD that does not respond to standard treatments, immunosuppressant drugs are now being used for long-term treatment. All of these drugs suppress actions of the immune system and thereby its inflammatory response that causes ulcerative colitis and Crohn's disease. The two most common immuno-suppressants used for IBD are azathioprine and mercaptopurine. Other immunosuppressants being investigated for IBD and showing promising results in promoting remission include cyclosporine and methotrexate.

Metronidazole is an antibiotic used for infections caused by anaerobic bacteria and is useful for people with Crohn's disease. Other antibiotics used for Crohn's disease include trimethoprim/sulfamethoxazole, ciprofloxacin, and tetracycline.

Of some promise is a genetically engineered antibody that acts against tumor necrosis factor (TNF), a major factor in the inflammatory process that causes IBD. Recent results show a reduction in disease activity and improving symptoms in both Crohn's disease and ulcerative colitis. A similar drug, cA2, is also showing promising results against Crohn's disease.

Asthma is a chronic lung disease and causes breathing problems. Asthma medicines keep the air tubes in the lungs open. There are two groups of asthma medicines: bronchodilators and anti-inflammatory active agents. Inhaled corticosteroids are important in therapy.

Chronic obstructive pulmonary disease (COPD) is defined as obstruction of the airways of the lungs of a persistent non-reversible nature. It is a generic term that includes chronic obstructive bronchitis, emphysema and asthmatic bronchitis.

While advances have been made in treating of COPD, there are no quick cures and response to therapy is often marginal, with indications that those individuals who have an advanced stage of the disease have marginal chances for survival. With this said, there are indications that new procedures are on the horizon that would make this outcome not as deadly as it is now. These include new bronchodilators and anti-inflammatory agents as well as lung transplantation and lung reduction surgery.

The current standard for treating COPD is Atrovent, which is a drug that has to be taken 4 times daily.

Psoriasis is a common condition affecting the skin. It causes red, scaly patches. In addition it can affect the joints, nails and eyes. Although the exact cause is unknown, psoriasis is believed to be related to faulty signals sent by the body's immune system. It has a genetic component that makes certain people more likely to develop it.

Treatments include: Moisturising creams and ointments, oils for the bath, creams, ointments, lotions and shampoos based on tar, vitamin D, salicylic acid, sun shine, stronger medications, eg methotrexate, and mild steroid creams and ointments, used for short periods, for psoriasis affecting the face or body folds.

The AIDS epidemic, cancer chemotherapy and organ transplantation have significantly increased the number of patients with impaired immune systems who are suffering from severe opportunistic infections including pneumonia caused by the fungus Pneumocystis carinii. P. carinii pneumonia (PCP), which is a serious disease with high prevalence, constitutes the major cause of death in patients with AIDS. Current treatment of the disease with trimethoprim (TMP structure below), a nonclassical inhibitor of dihydrofolate reductase (DHFR), in combination with a sulfonamide is still the standard therapy for PCP. Severe side-effects associated with sulfa drugs often lead to discontinuation of therapy. Inhaled aerosolised/nebulised pentamidine is used for prophylaxis. When applied systematically pentamidine exhibits a considerable toxicity. Trimetrexate (TMQ) and piritrexim (PTX), two new lipophilic agents originally developed against cancer are now used in clinic as second-line therapy (structures below). Although TMQ and PTX are both potent inhibitors of DHFR from P. carinii, they are not selective and inhibit the mammalian enzyme even more efficiently. The clinical use of TMQ and PTX is therefore limited due to their systemic host toxicity and require an expensive co-therapy with the rescue agent leucovorin (5-formyl-tetrahydrofolate). Leucovorin, a classical folate cofactor for one-carbon metabolism, is taken up via active transport only by mammalian cells and thereby reverses toxicity associated with the lipophilic DHFR inhibitors. Today considerable research efforts are devoted to the identification of more selective and potent DHFR inhibitors with the overall goal to improve therapy and to minimise the adverse effects.

Rheumatoid arthritis is another inflammatory condition, the signs and symptoms of which include, pain and swelling in the smaller joints of your bands and feet, overall aching or stiffness of the joints and muscles, especially after sleep or after periods of rest, loss of motion of the affected joints, loss of strength in muscles attached to the affected joints, fatigue, which can be severe during a flare-up, low-grade fever, deformity of the joints as time goes on.

Medications for rheumatoid arthritis can relieve its symptoms. Nonsteroidal anti-inflammatory drugs can slow or halt its progression. Treatment with NSAIDs can, however, lead to such side effects as indigestion and stomach bleeding, as well as damage to the liver and kidneys, ringing in the ears (tinnitus), fluid retention, and high blood pressure. COX-2 inhibitors, which is a new class of NSAIDs may be less damaging to the stomach, but may have higher incidents of other side-effects than conventional NSAIDs. Corticosteroids reduce inflammation and slow joint damage. Disease-modifying antirheumatic drugs (DMARDs) are another group of drugs prescribed. Some commonly used DMARDs include hydroxychloroquine sulfate (Plaquenil), gold compounds (Ridaura, Solganal), sulfasalazine (Azulfidine) and minocycline (Minocin). Other forms of DMARDs include immunosuppressants and TNF blockers. Some of the commonly used immunosuppressants include methotrexate, leflunomide, azathioprine, cyclosporine and cyclophosphamide. These medications can have potentially serious side effects such as increased susceptibility to infection and disease.

Antifolate Compounds in the Treatment of Bacterial Infections.

Sulfonamides are structural analogues of p-aminobenzoic acid. They interfere with the early stages of folic acid synthesis by competitive inhibition of dihydropteroic acid synthetase, which condenses p-aminobenzoic acid with dihydropteroic acid. The sulfonamide may also be erroneously incorporated into the folic acid molecule to produce an inactive product. Bacterial cells synthesize folic acid, whereas mammalian cells use the preformed dietary vitamin, and this is the basis of the selective antibacterial action of sulfonamides.

Diaminopyrimidines, like trimethoprim and the antimalarial compound, pyrimethamine, act at a later stage on the same pathway by inhibiting dihydrofolate reductase, the enzyme that generates the active product, tetrahydrofolate, from dihydrofolate. The affinity of trimethoprim for DHFR of bacteria is several orders of magnitude higher than the affinity for the mammalian enzyme; similarly pyrimethamine has a very high affinity for the DHFR of malaria parasites.

Because sulfonamides and diaminopyrimidines act on the same metabolic pathway, they exhibit a strongly synergic interaction, at least in vitro. However, because tetrahydrofolate is reoxidized to dihydrofolate during the biosynthesis of thymidylic acid, diaminopyrimidines rapidly trap the vitamin in the unusable dihydrofolate form. Sulfonamides, in contrast, cut off the supply of dihydrofolate and act rather slowly because the folate pool becomes depleted only after several cell divisions. For this reason, if there is sufficient diaminopyrimidine present to halt tetrahydrofolate regeneration completely, the sulfonamide does not have an opportunity to contribute to the antibacterial action.

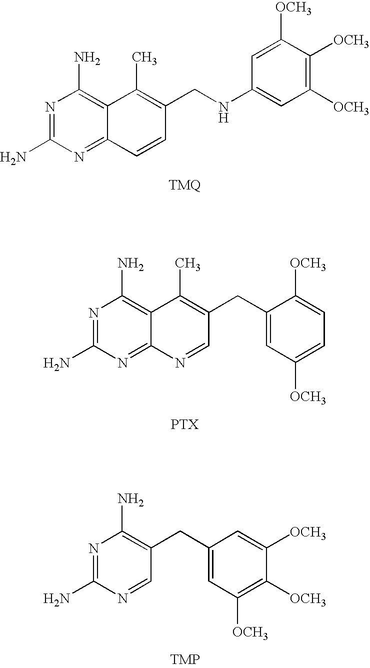

Certain ester analogues of the above DHFR inhibitors have been published in the academic literature. In particular Hallberg et al. (Chem. Pharm. Bull., 1998, 46, 591-601 and J. Med. Chem., 2000, 43, 3852-3 861) describes compounds of general formula I:

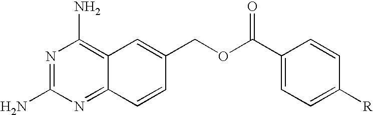

wherein R is hydrogen or a glutamic acid linked to the phenyl ring by an amide bond. However, the R=hydrogen variant showed a poor hydrolysis rate in in vitro assays comprising pig liver esterase, cholesterol esterase, human duodenal mucosa, human liver, rat liver and human leukocytes, respectively, leading the authors to conclude that “esters comprising a 2,4-diamino-pyrimidine ring are not suitable as soft drugs” (Chem. Pharm. Bull., 1998, 46, 591-601). The glutamic acid derivative exhibited a pharmacokinetic profile similar to methotrexate and was as expected relative inert to ester hydrolysis in vivo in rat (J. Med. Chem., 2000, 43, 3852-3861). The compounds of the present invention are however structurally different so the observed effects are unexpected.

### BRIEF DESCRIPTION OF THE INVENTION

We have now discovered that certain active isostenic-isoelectronic analogues of lipophilic DHFR structures tend to be deactivated by a fast hydrolytic metabolism in vivo. These DHFR inhibitors, consisting of an ester in the middle region, are thus more easily metabolised than the corresponding non-lipophilic, ester analogues of classic DHFR inhibitors and will in general be administered near the site of action following the criteria for the soft drug concept (Med. Res. Rev., 2000, 20, 58-101). The invention includes novel ester compounds. The invention further provides a new entry to efficient and safe treatment of diseases which can be therapeutically treated by immuno-modulating or cytostatic effective compounds, in particular DHFR inhibitors, either applied topically, orally or parenterally, or cancer forms being sensitive to methotrexate. IBD, i.e. ulcerative colitis and Crohn's disease, is a further indication that can be treated, and some other are colorectal cancer, cancer of the urinary bladder, the lung and other cancer types that may be reached from the “outside” of the body, psoriasis, PCP, other fungal (vaginal and others), protozoal and bacterial (pulmonary infections, urinary tract infections and others) infections, non-surgical abortions (intrauterin administration), asthma, or other serious pulmonary diseases, rheumatoid arthritis (other inflammatory conditions), may be treated, but can also be used as an agent non-rejecting liver transplantation, intestine transplantation. As a short-lived duration of exposure is sufficient, systemic treatment of e.g., rheumatoid arthritis or other inflammatory conditions, is possible as well. The compounds of the invention can also be used for treating nephritis, e.g. IgA nephritis.

Compounds of the present invention are either agonists or antagonists of a specific Melanocortin-receptor (MC-receptor) or of a number of MC-receptors, e.g. MC1, MC3, MC4 or/and MC5 receptors.

The MC-receptors belong to the class of G-protein coupled receptors, which are all built from a single polypeptide forming 7 transmembrane domains. Five such receptors types, termed MC1 , MC2, MC3, MC4 and MC5, have been described. The MC receptor's signalling is mainly mediated via cAMP but also other signal transduction pathways are known. They are distinctly distributed in the body.

MC-receptors are linked to a variety of physiological actions that are believed to be mediated by distinct subtypes of the MC-receptors. In many cases, however, it is not entirely clear which of the subtypes is responsible for the effect.

It has long been known that MSH-peptides may affect many different processes such as motivation, learning, memory, behaviour, inflammation, body temperature, pain perception, blood pressure, heart rate, vascular tone, brain blood flow, nerve growth, placental development, aldosterone synthesis and release, thyroxin release, spermatogenesis, ovarian weight, prolactin and FSH secretion, uterine bleeding in women, sebum and pheromone secretion, blood glucose levels, intrauterine foctal growth, as well as other events surrounding parturition (Eberle, AN: The melanotropins: Chemistry, physiology and mechanisms of action. Basel: Karger, Switzerland. 1998, ISBN 3-8055-4678-5; Gruber, and Callahan, Am. J. Physiol. 1989, 257, R681-R694; De Wildt et al., J. Cardiovascular Pharmacology. 1995, 25, 898-905), as well as inducing natriuresis (Lin et al., Hypertension. 1987, 10, 619-627).

It is also well known that the immunomodulatory action of α-MSH includes both immuno-stimulatory and immunosuppressive effects. Several studies have shown that α-MSH antagonizes the effects of pro-inflammatory cytokines such as IL-1α, IL-1 β, IL-6 and TNFα, and induces the production of the anti-inflammatory cytokine, IL-10 (for review see Catania & Lipton, 1993).

The invention provides novel compounds of the formula II:

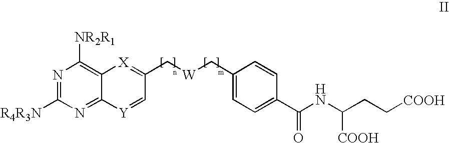

wherein R₁, R₂, R₃ and R₄ are independently hydrogen or a group that liberates the free amine in vivo, for example —CO-alkyl, preferably —CO—C₁-C₃ alkyl or pivalate; or —CO-haloalkyl, preferably —CO—C₁-C₃ haloalkyl, most preferably —CO—C₁-C₃ chloroalkyl;

wherein W is;

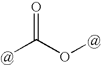

and @ denotes the points of attachment and wherein the ester can be located in either direction;

wherein n and m are independently 0-5;

wherein one but not both of X and Y can be nitrogen, or X is C-A and/or Y is C—B;

wherein A and B are independently selected from hydrogen, alkyl optionally substituted with a halogen, an electron donor group such as amino, alkylamino, dialkylamino or hydroxy, or an electron acceptor group such as nitro, cyano, trihaloalkyl or amido, alkoxy or halogen; and pharmacologically acceptable salts thereof;

Provided that, when R₁ to R₄ are hydrogen, both X and Y are C—H, n is 1 and —(CH₂)ₙ— is attached to the bridging oxygen of the ester group W, then m cannot be 0 or 1.

Preferably n is 3.

Preferably at least one of X and Y is nitrogen.

Most preferably, X is nitrogen.

In another preferred embodiment, X is preferably C-A and A is methyl and Y is C—B and B is hydrogen.

The term alkyl used herein means a straight or branched saturated hydrocarbon group having 1 to 4 carbon atoms.

The term alkoxy used herein means an —O-alkyl group.

The term trihaloalkyl as used herein includes trifluoroalkyl, trichloroalkyl tribromoalkyl and triiodoalkyl, preferably trifluoroalkyl and trichloroalkyl, and most preferably trifluoromethyl, trifluoroethyl, trichloromethyl and trichloroethyl.

The terms halo and halogen used herein include fluoro, chloro, bromo and iodo, preferably fluoro, chloro and bromo.

Although certain of the novel compounds may show decreased potency against pathogen or cellular DHFR relative to current inhibitors, they may exhibit somewhat better selectivity versus liver or other key DHFR species and, importantly, a more rapid metabolism to inactive metabolites in vivo. Accordingly, the compounds of the invention have utility in the treatment of diseases which can be therapeutically treated by immuno-modulating or cytostatic compounds, either applied topically, orally, rectally, or parenterally, or cancer forms being sensitive to methotrexate. Another utility are the treatments of IBD, i.e. ulcerative colitis and Crohn's disease. Asthma, Pneumocystis carinii pneumonia (PCP), psoriasis, rheumatoid arthritis (other inflammatory conditions), colorectal cancer, cancer of the urinary bladder, the lung and other cancer types that may be reached from the “outside” of the body, inflammatory conditions caused by bacterial, fungal (vaginal and others) or protozoal infections, non-surgical abortions (intrauterin administration), liver transplantation, or other serious pulmonary diseases may be treated, especially in immuno-compromised individuals.

A further aspect of the invention thus provides compounds of general formula II for use in therapy, such as use in the manufacture of a medicament for the treatment of disorders requiring the inhibition of DHFR.

The compounds of general formula II mediate their effects through one or several of the MC-receptors and thus a further aspect of the invention provides compounds that can be used for treating diseases and conditions involving the MC-receptors.

Compounds of formula II and/or their pharmaceutically acceptable salts have valuable pharmacological properties, making them useful for the treatment of inflammation such as inflammations related to the production of nitric oxide, inflammation related to increased amounts (upregulated amounts) of inducible nitric oxide synthase, inflammation related to activation of transcriptional activators, inflammation related to nuclear factor kappa beta, inflammation related to macrophages, neutrophils, monocytes, keratinocytes, fibroblasts, melanocytes, pigment cells and endothelial cells, inflammation related to increased production and/or release of inflammatory cytokines, such as e.g. interleukins, in particular interleukin 1 (IL-1), interleukin 6 (IL-6) and tumor necrosis factor α (TNF-α).

In the present specification, increased production refers to increased formation, increased release, or increased amount of an endogenous compound locally, regionally or sytemically in a patient compared to the amount of said endogenous compound in a healthy individual. In the present specification, upregulated refers to an increased activity or amount of the compound compared with that in a healthy individual.

In the present specification, decreased production refers to decreased formation, decreased release, or decreased amount of an endogenous compound in a patient compared to the amount of said endogenous compound in a healthy individual. In the present specification, downregulated refers to a decreased activity or amount of the compound compared with that in a healthy individual.

In particular, positive treatment effects or preventive effects may be seen in conditions where inflammation or an inflammatory-like condition is caused by or being associated with one or more of the following: allergy, hypersensitivity, bacterial infection, viral infection, inflammation caused by toxic agent, fever, autoimmune disease, radiation damage by any source including UV-radiation, X-ray radiation, γ-radiation, α- or β-particles, sun burns, elevated temperature or mechanical injury. Moreover, inflammation due to hypoxia, which is optionally followed by reoxygenation of the hypoxic area, is typically followed by severe inflammation, which condition may be positively affected by treatment with a compound of the invention.

In very specific embodiments of the invention, a compound of the invention may be administered for the prevention or therapeutic treatment of inflammatory diseases of the skin (including the dermis and epidermis) of any origin, including skin diseases having an inflammatory component. Specific examples of this embodiment of the invention include treatment of contact dermatitis of the skin, sunburns of the skin, burns any cause, and inflammation of the skin caused by chemical agents, psoriasis, vasculitis, pyoderma gangrenosum, discoid lupus erythematosus, eczema, pustulosis palmo-plantaris, and phemphigus vulgaris.

According to one aspect of the invention a compound of formula II or a pharmacologically acceptable salt thereof for the treatment of an inflammatory disease in the abdomen, including an abdominal disease having an inflammatory component. Specific examples of the treatment of such a disease with a compound of the invention are gastritis, including one of unknown origin, gastritis perniciosa (atrophic gastritis), ulcerous colitis (colitis ulcerosa), morbus Crohn, systemic sclerosis, ulcus duodeni, coeliac disease, oesophagitis and ulcus ventriculi.

According to one aspect of the invention administration of a compound of formula II or a pharmacologically acceptable salt thereof for the treatment of systemic or general and/or local immunological diseases, including those of an autoimmune nature, and other inflammatory diseases of a general nature. Specific examples include treatment of rheumatoid arthritis, psoriatic arthritis, systemic sclerosis, polymyalgia rheumatica, Wegener's granulomatosis, sarcoidosis, eosinophilic fasceitis, reactive arthritis, Bechterew's disease, systemic lupus erythmatosus, arteritis temporalis, Behcet's disease, morbus Burger, Good Pastures' syndrome, eosinophilic granuloma, fibromyalgia, myositis, and mixed connective tissue disease. Included therein is also arthritis, including arthritis of unknown origin.

According to one aspect of the invention administration of a compound of formula II or a pharmacologically acceptable salt thereof for the treatment of a disease of the peripheral and/or central nervous system related to inflammation. Included in this aspect of the invention is the treatment of cerebral vasculitis, multiple sclerosis, autoimmune ophthalmitis and polyneuropathia. Comprised by the invention is also the administration of a compound of the invention for the treatment of an inflammation of the central nervous system to prevent apoptotic cell death. Moreover, as some of the compounds of the invention show a distinct ability to induce nerve regeneration, positive treatment effects are often seen in central nervous system diseases involving damage of cells in this region. This aspect of the invention also includes treatment of traumatic injuries to the central nervous system, brain edema, multiple sclerosis, Alzheimer's disease, bacterial and viral infections in the central nervous system, stroke, and haemorrhagia in the central nervous system.

According to one aspect of the invention administration of a compound of formula II or a pharmacologically acceptable salt thereof for the treatment of diseases of the eye and tear glands related to inflammation. Specific examples of such diseases comprise anterior and posterior uveitis, retinal vasculitis, optic neuritis, optic neuromyelitis, Wegener's granulomatosis, Sjügren's syndrome, episcleritis, scleritis, sarcoidosis affecting the eye and polychondritis affecting the eye.

According to one aspect of the invention administration of a compound of formula II or a pharmacologically acceptable salt thereof for the treatment of diseases of the ear related to inflammation, specific examples of which include polychondritis affecting the ear and external otitis.

According to one aspect of the invention administration of a compound of formula II or a pharmacologically acceptable salt thereof for the treatment of diseases of the nose related to inflammation, specific examples of which are sarcoidosis, polychondritis and mid-line granuloma of the nose.

According to one aspect of the invention administration of a compound of formula II or a pharmacologically acceptable salt thereof for the treatment of diseases related to inflammation of the mouth, pharynx and salivary glands. Specific examples include Wegener's granulomatosis, mid-line granuloma, Sjügren's syndrome and polychondritis in these areas.

According to one aspect of the invention administration of a compound of formula II or a pharmacologically acceptable salt thereof for the treatment of diseases related to inflammation in the lung. Specific examples include treatment of idiopathic alveolitis, primary pulmonary hypertension, bronchitis, chronic bronchitis, sarcoidosis, alveolitis in inflammatory systemic disease, pulmonary hypertension in inflammatory systemic disease, Wegener's granulomatosis and Good Pastures' syndrome.

According to one aspect of the invention administration of a compound of formula II or a pharmacologically acceptable salt thereof for the treatment of diseases related to the inflammation of the heart. Specific examples include treatment of pericarditis, idiopathic pericarditis, myocarditis, Takayasus' arteritis, Kawasaki's disease, coronary artery vasculitis, pericarditis in inflammatory systemic disease, myocarditis in inflammatory systemic disease, endocarditis and endocarditis in inflammatory systemic disease.

According to one aspect of the invention administration of a compound of formula II or a pharmacologically acceptable salt thereof for the treatment of diseases related to inflammation of the liver. Specific examples include treatment of hepatitis, chronic active hepatitis, biliary cirrhosis, hepatic damage by toxic agents, interferon induced hepatitis, hepatitis induced by viral infection, liver damage induced by anoxia and liver damage caused by mechanical trauma.

According to one aspect of the invention administration of a compound of formula II or a pharmacologically acceptable salt thereof for the treatment of diseases related to inflammation of the pancreas. Specific examples include treatment (and prevention) of diabetes mellitus, acute pancreatitis and chronic pancreatitis.

According to one aspect of the invention administration of a compound of formula II or a pharmacologically acceptable salt thereof for the treatment of diseases related to the inflammation of the thyroidea. Specific examples of these embodiments of the invention include treatment of thyreoiditis, autoimmune thyreoiditis and Hashimoto's thyreoiditis.

According to one aspect of the invention administration of a compound of formula II or a pharmacologically acceptable salt thereof for the treatment of diseases related to inflammation of the kidney. Specific examples include treatment of glomerulonephritis, glomerulonephritis in systemic lupus erythematosus, periarteritis nodosa, Wegener's granulomatosis, Good-Pastures' syndrome, HLAb27 associated diseases, IgA nephritis (IgA=Immunoglobulin A), pyelonephritis, chronic pyelonephritis and interstitial nephritis.

According to one aspect of the invention administration of a compound of formula II or a pharmacologically acceptable salt thereof for the treatment of diseases related to the inflammation of the joints. Specific examples include treatment of Bechterew's disease, psoriatic arthritis, rheumatoid arthritis, arthritis in colitis ulcerosa, arthritis in morbus Crohn, affection of joints in systemic lupus erythematosus, systemic sclerosis, mixed connective tissue disease, reactive arthritis, Reiter's syndrome. Moreover, included in this embodiment of the invention is treatment of arthrosis of any joint, in particular arthrosis of finger joints, the knee and the hip.

According to one aspect of the invention administration of a compound of formula II or a pharmacologically acceptable salt thereof for the treatment of diseases related to the inflammation of blood vessels. Specific examples include treatment of arteritis temporalis, perarteritis nodosa, arteriosclerosis, Takayasus' arteritis and Kawasaki's disease.

According to one aspect of the invention administration of a compound of formula II or a pharmacologically acceptable salt thereof for the treatment of inflammation related to infections of any origin. Specific examples include treatment of inflammation secondary to infection caused by virus, bacteria, helminths and protozoae.

According to one aspect of the invention administration of a compound of formula II or a pharmacologically acceptable salt thereof for the treatment of inflammations related to trauma and/or tissue injury of any origin.

The activities of compounds of the invention against various cellular and pathogen DHFR are measured by conventional assays, such as those described in Antimicrob. Agents Chemother., 1991, 35, 1348-1355 and Antimicrob. Agents Chemother., 1993, 37, 1914-1923. Preferred compounds will typically show a show IC50 for the target DHFR, and under certain circumstances in the matter of treating P. carinii, show, in conjunction with high selectivity index between cellular DHFR (for example rat liver DHFR) and the relevant pathogen DHFR. As a reference point it should be noted that conventional DHFR inhibitors such as PTX and TMQ have selectivity indexes of the order 0.13-0.19, which is generally exceeded in the compounds of the invention.

The free bases of formula II may be converted to their therapeutically active acid addition salts, which form an additional aspect of the invention. Appropriate pharmaceutically acceptable salts of the compounds of general formula II include salts of organic acids, especially carboxylic acids, including but not limited to acetate, trifluoroacetate, lactate, gluconate, citrate, tartrate, maleate, malate, pantothenate, isethionate, adipate, alginate, aspartate, benzoate, butyrate, digluconate, cyclopantanate, glucoheptanate, glycerophosphate, oxalate, heptanoate, hexanoate, fumarate, nicotinate, palmoate, pectinate, 3-phenylpropionate, picrate, pivalate, proprionate, lactobionate, pivolate, camphorate, undecanoate and succinate, organic sulphonic acids such as methanesulphonate, ethanesulphonate, 2-hydroxyethane sulphonate, camphorsulphonate, 2-napthalenesulphonate, benzenesulphonate, p-chlorobenzenesulphonate and p-toluenesulphonate; and inorganic acids such as hydrochloride, hydrobromide, hydroiodide, sulphate, bisulphate, hemisulphate, thiocyanate, persulphate, phosphoric and sulphonic acids. Hydrochloric acid salts are convenient. The salt may also be hydrated to various degrees, e.g. mono-, di- or tri-hydrates.

Conversely, the salt form may be converted into the free base form by treatment with alkali.

The compounds of the invention are particularly suited to topical administration, such as pulmonary, dermally, optically, vaginally, nasally, transdermally but may also be administered orally, rectally, or parenterally, for instance orally in a bioadhesive composition to adhere to the gastro-intestinal tract or parenterally as intramuscularly, intraperitoneally, intravenously or epidurally. The compounds may be administered alone, for instance in a capsule, but will generally be administered in conjunction with a pharmaceutically acceptable carrier or diluent. The invention extends to methods for preparing a pharmaceutical composition comprising bringing a compound of formula II or its pharmaceutically acceptable salt in conjunction or association with a pharmaceutically acceptable carrier, adjuvant, excipient or vehicle.

The term topical herein means any application on the outside of the body but also applies to the topical administration on the mucous membranes of the gastro-intestinal tract, such as by means of a mucoadhesive composition adhering to e.g., the intestines where it serves its therapeutically effect.

Oral formulations are conveniently prepared in unit dosage form, such as capsules or tablets, employing conventional carriers or binders such as magnesium stearate, chalk, starch, lactose, wax, gum or gelatine. Liposomes or synthetic or natural polymers such as HPMC or PVP may be used to afford a sustained release formulation. Alternatively the formulation may be presented as a nasal or eye drop, syrup, gel or cream comprising a solution, suspension, emulsion, oil-in-water or water-in-oil preparation in conventional vehicles such as water, saline, ethanol, vegetable oil or glycerine, optionally with flavouring agent and/or preservative and/or emulsifier. Any formulation may contain 0.5 to 99.5% by weight of the therapeutically active compound.

### EXAMPLES

The following examples are intended to illustrate but not to limit the scope of the invention, although the compounds named are of particular interest for the intended purposes. These compounds have been designated by a number code, a:b, where a means the number of example, wherein the preparation of the compound is described, and b refers to the order of the compound prepared according to that example. Thus example 1:2 means the second compound prepared according to example 1.

The structures of the compounds were confirmed by IR, NMR, MS and elementary analysis. When melting points are given, these are uncorrected.

Methods of Preparation

The compounds exemplified below can be prepared by the general procedure outlined in schemes 1 and 2.
Scheme 1

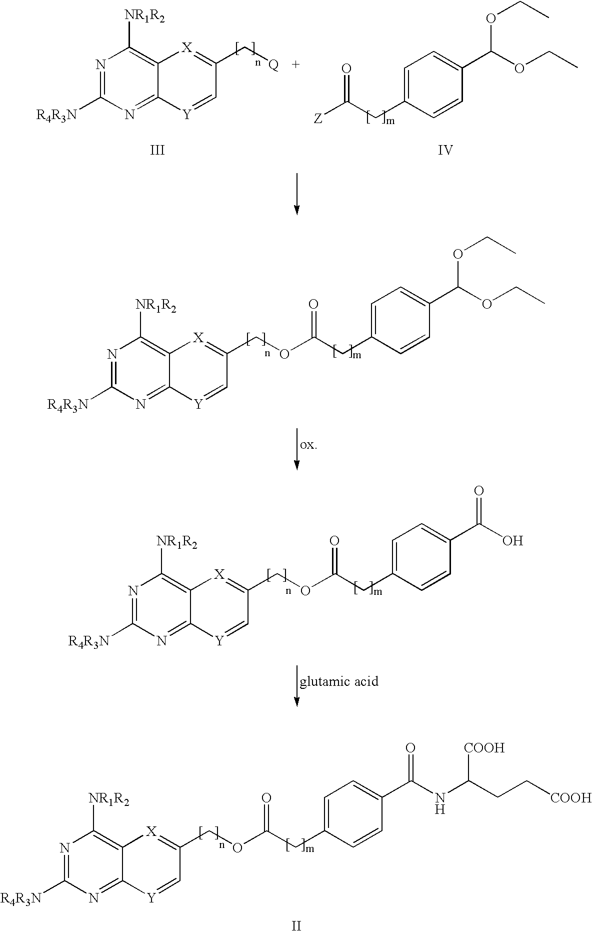

A compound of formula III wherein R₁ to R₄, n, X and Y are as defined above and wherein Q is OH or a halogen atom, for example Br, is reacted with a compound of formula IV wherein m is as defined above and wherein Z is halogen, for example Cl, or OH when Q is OH, and Z is OH when Q is halogen, using standard esterification methods well known in the art. Oxidation of the acetal protected aldehyde on the resulting ester followed by coupling with glutamic acid according to methods known in the art affords a compound of formula II (Scheme 1).
Scheme 2

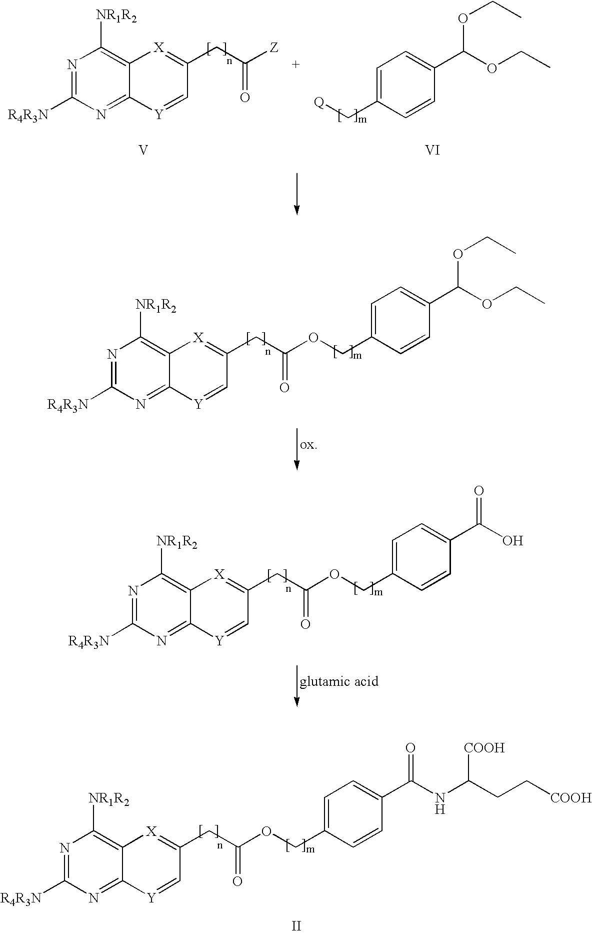

A compound of formula V wherein R₁ to R₄, n, X and Y are as defined above and wherein Z is halogen, e.g. Cl, or OH, is reacted with a compound of formula VI wherein m is as defined above and wherein Q is OH when Z is halogen and Q is OH or halogen, e.g. Br, when Z is OH, using standard esterification methods well known in the art. Oxidation of the acetal protected aldehyde on the resulting ester followed by coupling with glutamic acid according to methods known in the art affords a compound of formula II (Scheme 2).

If R₁ to R₄ are hydrogen, the free amines may be protected with a suitable protecting group during the esterification step. The protecting groups can be removed according to methods known per se.

Compound of formula IV and VI are commercially available or synthesised according to methods known in the art.

Compounds of formula III and V may be prepared according to the following scheme:

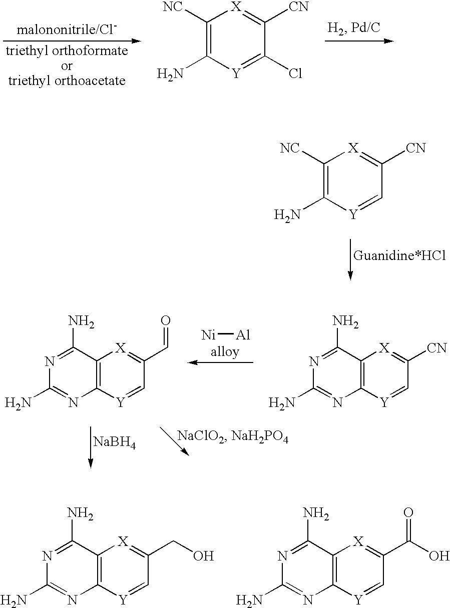

(From the thesis by Malin Graffner-Nordberg “Approaches to Soft Drug Analogues of Dihydrofolate Reductase Inhibitors” Uppsala University 2001, ISBN 91-554-5017-2).

When R1 to R4 are not hydrogen, the free amine groups can be converted using methods known in the art.

Compounds of formula III and V wherein X and Y represent C-A and C—B can be prepared from the corresponding 6-nitro compound, which is commercially available, via reduction, diazotisation and a choice of reagent to introduce either —OH or —CN, whence further functionalisation can be effected, all of which methods are known per se.

Functionalisation of a group ═C—CH₃ as produced in the scheme above by reaction with triethyl orthoacetate to produce other compounds in which A or B is other than methyl may be carried out by methods known per se and/or as described in Comprehensive Organic Transformation by Richard C. Larock, 2 Bd”; John Wiley New York, 1999, and references therein.

To produce starting materials by the schemes above in which n, for example, is 0 so that the ring is attached directly to a hydroxyl group, corresponding compounds wherein an amino group, which can if desired be produced from a corresponding compound containing a nitro group, may be diazotised via nitrous acid and then treated with a source of hydroxyl ions to produce a nuclear —OH group.

To produce starting materials by the schemes above in which n is from 1-5, where they are not already produced, chain lengthening reactions may be performed by methods already known in the art. These may include conversion to halide compounds followed by Wurtz coupling or by use of Grignard reactions with a suitable adduct, followed by work-up, reduction if appropriate, and information of the desired starting material.

Acid halides of formula V can be synthesised from the corresponding carboxylic acid using methods known in the art. Halides of formula III can be synthesised from the corresponding alcohol using methods known in the art.

Thin-layer chromatography (TLC) was performed by using aluminum sheets precoated with silica gel 60 F254 (0.2 mm) type E; Merck. Chromatographic spots were visualized by UV light, or by an acidic ethanolic solution of 2,4-dinitrophenylhydrazine. Column chromatography was conducted on silica gel S (0.032-0.063 mm; Riedel-deHaén), silica gel 60 (0.040-0.063 mm; E. Merck), unless otherwise noted. Preparative TLC was performed on glass sheets precoated with silica gel 60 F₂₅₄ (2.0 mm; B. Merck). Melting points (uncorrected) were determined in open glass capillaries on an Electrothermal apparatus. Infrared (M) spectra were recorded on a Perkin-Elmer 1605 FT-IR spectrophototmeter and are recorded in νₘₐₓ (cm⁻¹). The elemental analyses were performed by Mikro Kemi AB, Uppsala, Sweden. All commercial chemicals were used without further purification.

General Procedure for the Synthesis of Compounds 1:1-1:4 and 2:1-2:3.

The esterification procedures may be carried out using methods well known in the art. These may readily be optimised for any given set of reactants by simple experiments. For example, for an esterification in which an alcohol of formula III or VI is reacted with a carboxylic acid of formula IV or V, reaction may be carried out by dissolving the alcohol in acetonitrile and beating to reflux when necessary. The carboxylic acid is then added and the mixture stirred for 24-48 hours. Where esterification is carried out using an acid halide of formula IV or V, such as the chloride, the reaction may be carried out in a solvent such as THF or DMF. Where esterification is carried out using a halide of formula III or VI, such as the bromide, reaction may be carried out in a solvent such as DMF. The crude product is purified by chromatography where necessary. Oxidation of the acetal protected aldehyde followed by coupling with glutamic acid gives the products. Purification by flash chromatography gives the pure product.

List of Compounds

| | | |
|---|---|---|
| 1:1 | 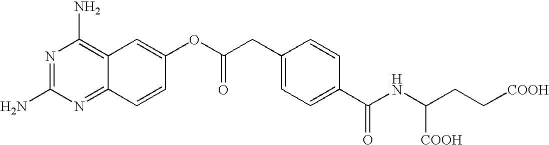 | 2-[4-(2,4-Diamino- quinazolin-6- yloxycarbonylmethyl)- benzoylamino]- pentanedioic acid |
| 1:2 | 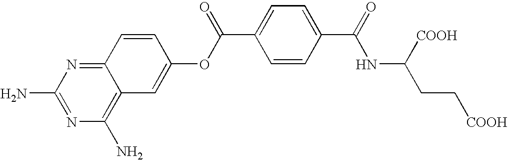 | 2-[4-(2,4-Diamino- quinazolin-6- yloxycarbonyl)- benzoylamino]- pentanedioic acid |
| 1:3 | 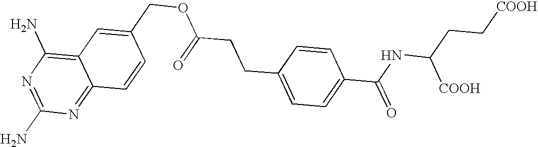 | 2-{4-[2-(2,4-Diamino- quinazolin-6- ylmethoxycarbonyl)-ethyl]- benzoylamino}- pentanedioic acid |
| 1:4 | 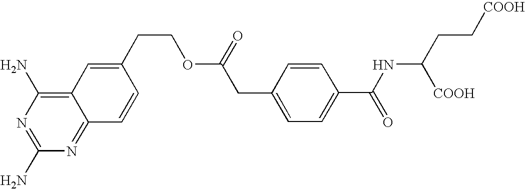 | 2-{4-[2-(2,4-Diamino- quinazolin-6-yl)- ethoxycarbonylmethyl]- benzoylamino}- pentanedioic acid |
| 2:1 | 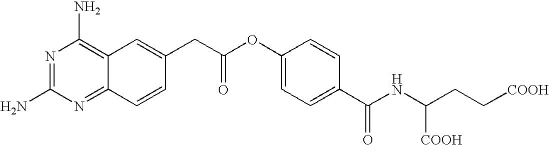 | 2-{4-[2-(2,4-Diamino- quinazolin-6-yl)-acetoxy]- benzoylamino}- pentanedioic acid |
| 2:2 | 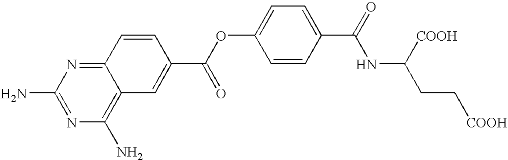 | 2-[4-(2,4-Diamino- quinazoline-6- carbonyloxy)- benzoylamino]- pentanedioic acid |
| 2:3 | 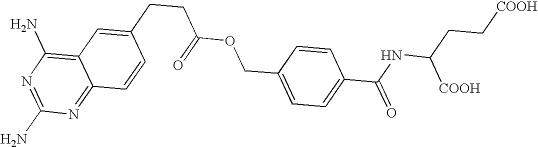 | 2-{4-[3-(2,4-Diamino- quinazolin-6-yl)- propionyloxymethyl]- benzoylamino}- pentanedioic acid |

### Example 2

This example describes the biological tests performed with the compounds of formula (I) and their therapeutically active acid addition salts.

Test 1. Affinity for the MC1-Receptor

The binding assay was carried out essentially as described by Lunec et at.; Melanoma Res. 1992; 2; 5-12 using I²⁵-NDP-αMSH as ligand.

Test 2. Affinity for the MC3-Receptors, the MC4-Receptors and the MC5-Receptors

The binding assays were carried out essentially as described by Szardenings et al., J. Biol. Chem. 1997; 272, 27943-27948 and Schiöth et al., FEBS Lett. 1991; 410; 223-228 using I¹²⁵-NDP-αMSH as ligand.

Essentially, the affinity of the compounds for the different melanocortin receptors were determined by using either insect cells (Sf9) or COS cells, which were transfected with recombinant human MC3, MC4 or MC5 receptors. For the determination of the affinity for the MC1 receptor, B16 mouse melanoma cells were used, which endogenously express the (mouse) MC1 receptor.

The compounds were tested at different concentrations for their ability to displace a ¹²⁵I-labelled NDP-MSH from the respective receptor. Incubation was performed in 96-well plates, using 50,000 cells/well (Sf9 or COS cells) up to 200,000 cells/well (mouse melanoma cells).

The test compound or standard (NDP-MSH) was added in an appropriate concentration (generally between 10⁻⁴ M and 10⁻¹² M) together with labelled tracer (approx. 50,000 cpm/well) and incubated for 2 hours (at room temperature for Sf9 cells and at +37° C. for COS cells and mouse melanoma cells).

After the incubation, the cells were washed twice to get rid of the excess tracer and compound, and the cells were lysed with 0.1 M NaOH. The lysate was counted in a gamma-counter, binding was calculated and the affinity determined.

Test 3. cAMP Assay

Essentially, the effects of the compounds are tested in vitro for their ability to stimulate the production of cAMP. The cells used are mouse melanoma B16 cells, which endogenously express the (mouse) MC1 receptor, and Chinese Hamster Ovary (CHO) cells stably transfected with the human MC4 receptor, the mouse MC3 receptor or the mousse M5 receptor.

Adherent cells are cultured in 96-well plates overnight and are challenged with various concentrations of test compound, for 20 minutes at +37° C. The concentration of intracellular cAMP is then determined on cell lysates by the use of a enzyme immunoassay (EIA) (RPN225, Amersham Biosciences). The cAMP in the cell lysates are allowed to bind to a limited number of binding sites on a cAMP-specific antibody for 2 hours before a fixed quantity of peroxidase-labelled cAMP, which will compete for the binding sites, are added for 1 hour. Bound antibodies are detected by the addition of an enzyme substrate that changes colour by the action of the peroxidase. After 1 hour 1 M sulphuric acid is added to stop the reaction. The optical densities of the developed coloured solutions are determined spectrophotometrically at 450 mm and the concentrations of cAMP in the samples are calculated from a standard curve.

Test 4. DHFR Assay

The Pneumocystis Carnii DHFR used was prepared and purified according to literature methods (Broughton & Qeener, Antimier. Agents and Chemother., 1991, 35 (7) 1348-1355). The spectrophotometric assay was conducted essentially according to method described by Allegra et al (Allegra et al, J. Exp. Med., 1987, 165, 926-931).

Test 5. IBD model

Dextran Sodium Sulphate-Induced Colitis in Mice

The Dextran sodium sulphate (DSS) model is considered to be a relevant model to study mechanisms involved in IBD in humans. Today there are several hundred articles published on the model and its response to different drugs. The immunomodulatory drug cyclosporin, given therapeutically, reduce disease activity in the DSS model in mice [Murthy S N S, Cooper H S, Shim H, Shah R S, Ibrahim S A, Sedergran D J. Treatment of dextran sulfite sodium-induced murine colitis by intracolonic cyclosporin. Dig Dis Sci 1993;38(9):1722-1734]. The DSS moodel has been evaluated by others [Cooper H S, Murthy S N S, Shah R S, Sedergran D J. Clinicopathologic study of dextran sulfate sodium experimental murine colitis. Lab Invest 1993;69(2):238-2491.].

Colonic inflammation is induced by oral administration of DSS in the drinking water. An induction period of 7-10 days with DSS is followed by a treatment period of 5-10days where DSS administration is continued and drugs or control substances are given. Parameters recorded at autopsy are body weight, spleen weight, diarrhea (wet/dry fecal weight), colon length and the bistopathological appearance of the colonic tissue.

1. A compound of general formula II 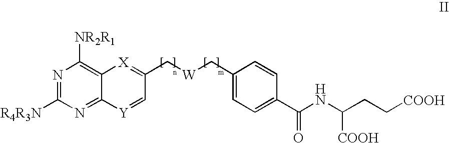
wherein R₁, R₂, R₃ and R4 are independently hydrogen or a group that liberates the free amine in vivo, for example —CO-alkyl, preferably-CO—C₁-C₃ alkyl or pivalate; or —CO-haloalkyl, preferably —CO—C₁-C₃ haloalkyl, most preferably —CO—C₁-C₃ chloroalkyl;
wherein W is; 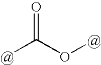
and @ denotes the points of attachment and wherein the ester can be located in either direction;
wherein n and m are independently 0-5;
wherein one but not both of X and Y can be nitrogen, or X is C-A and/or Y is C—B;
wherein A and B are independently selected from hydrogen, alkyl optionally substituted with a halogen, amino, alkylamino, dialkylamino, hydroxy, nitro, cyano, trihaloalkyl, amido, alkoxy or halogen; and pharmacologically acceptable salts thereof;
provided that when R₁ to R₄ are hydrogen, both X and Y are C—H, n is 1 and —(CH₂)ₙ₋ is attached to the bridging oxygen of the ester group W, then m cannot be 0 or 1.

2. A compound as claimed in claim 1 wherein X is C-A and Y is C—B.

3. A compound having one of the following formulae.

| | | |
|---|---|---|
| 1:1 | 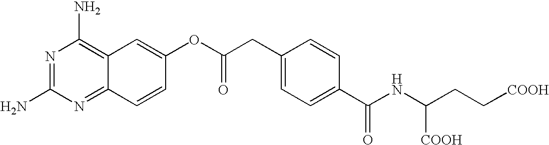 | 2-[4-(2,4-Diamino- quinazolin-6- yloxycarbonylmethyl)- benzoylamino]- pentanedioic acid |
| 1:2 | 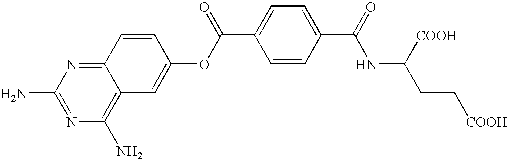 | 2-[4-(2,4-Diamino- quinazolin-6- yloxycarbonyl)- benzoylamino]- pentanedioic acid |
| 1:3 | 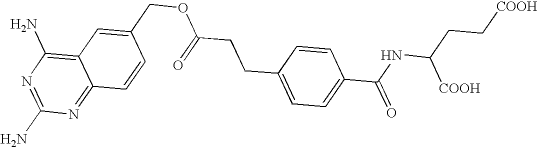 | 2-{4-[2-(2,4-Diamino- quinazolin-6- ylmethoxycarbonyl)-ethyl]- benzoylamino}- pentanedioic acid |
| 1:4 | 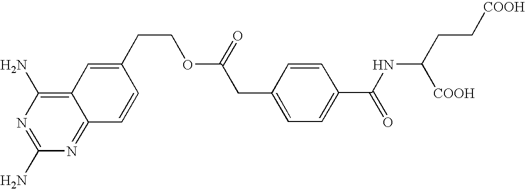 | 2-{4-[2-(2,4-Diamino- quinazolin-6-yl)- ethoxycarbonylmethyl]- benzoylamino}- pentanedioic acid |
| 2:1 | 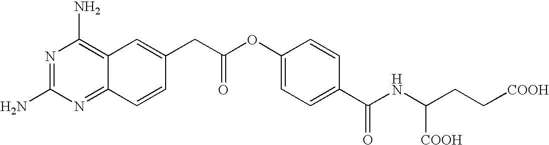 | 2-{4-[2-(2,4-Diamino- quinazolin-6-yl)-acetoxy]- benzoylamino}- pentanedioic acid |
| 2:2 | 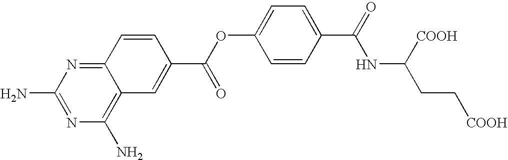 | 2-[4-(2,4-Diamino- quinazoline-6- carbonyloxy)- benzoylamino]- pentanedioic acid |
| 2:3 | 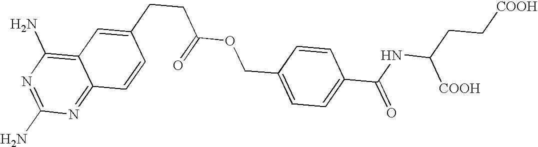 | 2-{4-[3-(2,4-Diamino- quinazolin-6-yl)- propionyloxymethyl]- benzoylamino}- pentanedioic acid |

4. A compound as claimed in claim 1, which additionally comprises a label preferably a radioactive label, or a toxic agent.

5. A pro-drug from which a compound as claimed in claim 1 can be formed in vivo.

6. A pharmaceutical composition comprising a compound as claimed in claim 1, together with one or more carriers, adjuvants or excipients.

7. (canceled)

8. A process for the production of a compound as claimed in claim 1, which comprises a reaction as, given in scheme 1 or 2.

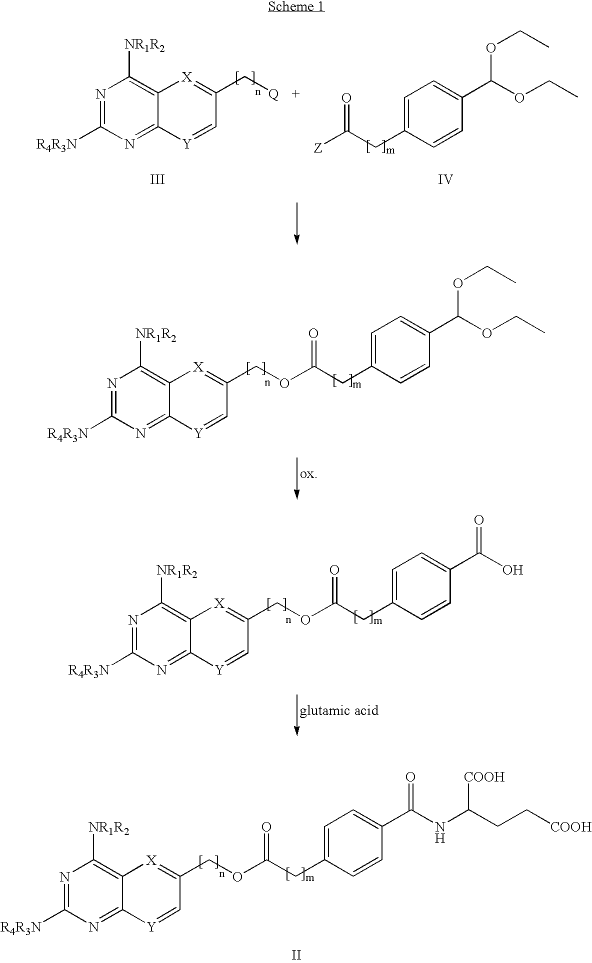

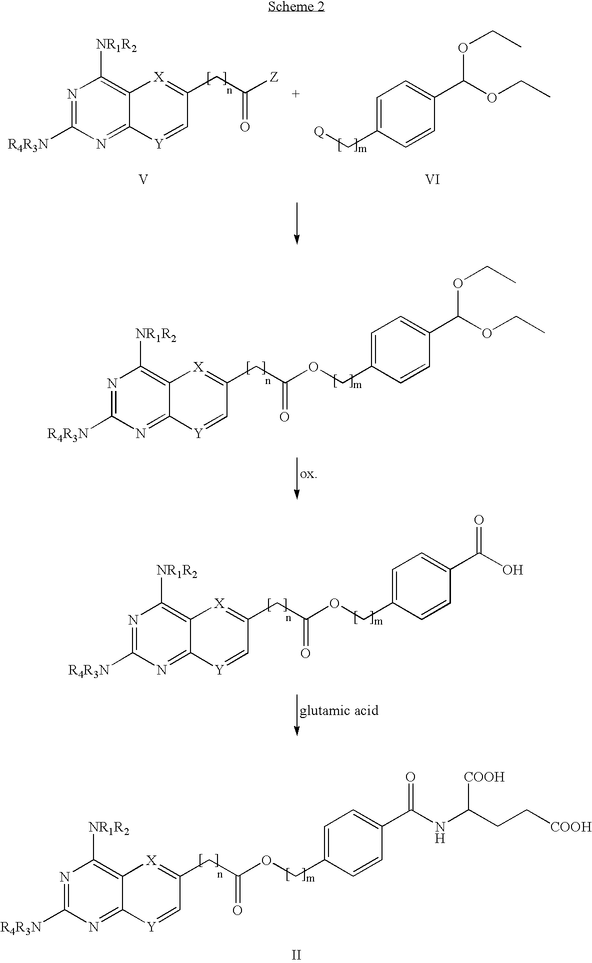

9-11. (canceled)

12. Method for treating a disease which can be therapeutically treated by immuno-modulating or cytostatic compounds, in particular dihydrofolate reductase inhibitors, either applied topically, orally or parenterally, or cancer forms being sensitive to methotrexate, IBD, i. e. ulcerative colitis and Crohn's disease, colorectal cancer, asthma, or other serious pulmonary diseases, PCP, psoriasis, inflammations caused by bacteria, fungi, protozoa, rheumatoid arthritis as well as other inflammatory conditions, cancer of the urinary bladder, the skin, the lung and other cancer types that are reachable by topical application, non-surgical abortions (intrauterin administration), liver and intestine transplantations by preventing immunogenic rejection reactions, whereby a therapeutically effective amount of at least one compound defined in claim 1 is administered for a time sufficient to substantially eliminate the signs and symptoms of such a disease

13. A method for the treatment of a disease, which is sensitive to an inhibition of dihydrofolate reductase comprising the administration of a therapeutically active amount of at least one compound as defined in claim 1.

14. A method according to claim 12 for the treatment of cancer forms being sensitive to methotrexate.

15. A method according to claim 12 for the treatment of IBD.

16. A method according to claim 12 for the treatment of PCP.

17. A method according to claim 12 for the treatment of psoriasis.

18. A method according to claim 12 for the treatment of rheumatoid arthritis.

19. A method according to claim 12 for the treatment of inflammation inflammations caused by fungal, protozoal and/or bacterial infections.

20. A method according to claim 12 for the treatment of asthma or pulmonary disease.

21. A method according to claim 12 for the treatment of liver or intestine transplantation by preventing immunogenic rejection reactions.

22. A method for the treatment of disease or condition related to the melanocortin system comprising the administration of a therapeutically active amount of at least one compound as defined in claim 1.

23. A pharmaceutical composition comprising a pro-drug as claimed in claim 5, together with one or more carriers, adjuvants or excipients.

24. A method for the treatment of inflammation, comprising administering a compound as defined in claim 1.

25. A method for the treatment of inflammation, comprising administering a pro-drug as defined in claim 5.
